(19) United States
(12) Patent Application Publication
(10) Pub. No.: US 2016/0243059 A1
(43) Pub. Date: Aug. 25, 2016

(54) PHARMACEUTICAL COMPOSITION FOR TREATING OR PREVENTING DEGENERATIVE BRAIN DISEASE COMPRISING MULTI-TARGETING COMPOUNDS

(71) Applicant: UNIST Academy-Industry Research Corporation, Ulsan (KR)

(72) Inventor: Mi Hee LIM, Ulsan (KR)

(73) Assignee: UNIST Academy-Industry Research Corporation, Ulsan (KR)

(21) Appl. No.: 14/967,376

(22) Filed: Dec. 14, 2015

(30) Foreign Application Priority Data
Feb. 25, 2015 (KR) 10-2015-0026202
Sep. 16, 2015 (KR) 10-2015-0130921

(51) Int. Cl. A61K 31/136; A23L 1/30

(52) U.S. Cl. CPC A61K 31/136; A23L 1/30; A23V 2002/00

(57) ABSTRACT

A pharmaceutical composition for treating or preventing a degenerative brain disease, the pharmaceutical composition including, as an active component, a multi-targeting compound for further improving treatment efficiency of a degenerative brain disease such as Alzheimer's disease is disclosed. The pharmaceutical composition includes a compound represented by Formula 1 or 2, wherein the compound of Formula 1 or 2 induces a gathering of amyloid-peptides in a manner of toxicity-free aggregation under conditions both in the presence and absence of metals such as copper and zinc, and also reacts with multiple targets of Alzheimer's diseases at once to inhibit toxicity thereof, the multiple targets including amyloid-peptide, metal-amyloid-peptide, a metal, and an activated oxidizing species. The pharmaceutical composition including the compound of Formula 1 or 2 may be utilized as a therapeutic agent or a health food in regard to a brain disease, including Alzheimer's disease.

### CROSS-REFERENCE TO RELATED PATENT APPLICATION

This application claims the benefit of Korean Patent Application No. 10-2015-0026202 filed on Feb. 25, 2015, and Korean Patent Application No. 10-2015-0130921 filed on Sep. 16, 2015 in the Korean Intellectual Property Office, the disclosure of which is incorporated herein in its entirety by reference.

### BACKGROUND OF THE INVENTION

1. Field of the Invention

One or more exemplary embodiments relate to a pharmaceutical composition for treating or preventing a degenerative brain disease, the pharmaceutical composition including, as an active component, a multi-targeting compound for further improving treatment efficiency of a degenerative brain disease such as Alzheimer's disease.

2. Description of the Related Art

Degenerative brain diseases are age-related diseases caused by dysfunction of brain nerve cells. Social interest has increased with a rapid increase in the aging population.

Degenerative brain diseases are classified according to major clinical symptoms and affected brain area, and include Alzheimer's disease, Parkinson's disease, Huntington's disease, multiple sclerosis, and amyotrophic lateral sclerosis.

Alzheimer's disease destroys central nerves, specifically nerves of the limbic system including forebrain, amygdala, hippocampus, and cortex such as cortical cortex. The limbic system includes regions related to learning, memorizing, thinking, behaving, and controlling emotion in the brain. In particular, a lack of neurotransmitters such as acetylcholine (ACh) is the most important indicator of Alzheimer's disease, and in this regard, recovering the lack of neurotransmitters is one of therapeutic goals for Alzheimer's disease.

Parasympathetic drugs for Alzheimer's disease can be divided into muscarine agonist or nicotine effectors, cholinesterase inhibitors (ChEIs), and drugs that indirectly control isolation of acetylcholine. Among them, many drugs have been developed and used as cholinesterase inhibitors ChEIs, e.g., tacrine, physostigmine, donepezil, rivastigmin, and memantin, have been developed and used.

Second-generation drugs, e.g., donepezil and rivastigmin, are more likely to have high concentrations in the central nervous system compared to first-generation drugs, e.g., tacrine, in terms of long reaction time, stability, and high permeability of blood-brain barrier (BBB) of the second-generation drugs. The first-generation drugs inhibit acetylcholinesterase (AChE), butyrylcholinesterase, and peripheral cholinesterase in a non-selective manner, whereas other several new drugs are known to have reduced peripheral side effects based on high selectivity to AChE. These drugs are also known to have mechanisms of inhibiting the breakdown of acetylcholine (ACh), resulting in an increase of concentrations of ACh in synapses.

Such drugs that have been conventionally used in the art for Alzheimer's disease cause serious side effects from long-term use, and thus, there is a need for the development of new drugs having similar or better pharmacological efficacy than that of conventional drugs and having fewer side effects.

### PRIOR ART DOCUMENT

### Patent Document

1. KR 2010-0009415 (published on Jan. 27, 2010)

### SUMMARY OF THE INVENTION

One or more exemplary embodiments include a pharmaceutical composition for treating or preventing a degenerative brain disease, the pharmaceutical composition including, as an active component, a multi-targeting compound for further improving treatment efficiency of a degenerative brain disease such as Alzheimer's disease.

One or more exemplary embodiments include a health food for preventing or improving a degenerative brain disease, the health food including, as an active component, a multi-targeting compound.

Additional aspects will be set forth in part in the description which follows and, in part, will be apparent from the description, or may be learned by practice of the presented embodiments.

According to one or more exemplary embodiments, there is provided a pharmaceutical composition for treating or preventing a degenerative brain disease, the pharmaceutical composition including, as an active component, a compound represented by Formula 1 or 2:

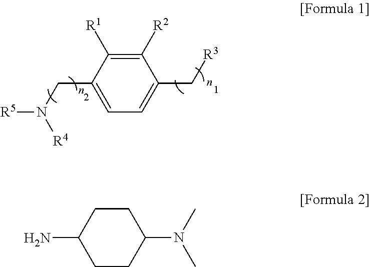

In Formula 1,

R¹ to R³ may be identical to or different from each other, and may each be independently at least one of a hydrogen, a halogen atom, a di(C₁-C₄ alkyl)amino group, and a carboxyl group,

R⁴ and R⁵ may be identical to or different from each other, and may each be independently at least one of a hydrogen and a C₁-C₄ alkyl group, and

n₁ and n₂ may be identical to or different from each other, and may each be independently an integer of 0 or 1.

According to one or more exemplary embodiments, there is provided a health food for preventing or improving a degenerative brain disease, the health food including, as an active component, the compound of Formula 1 or 2.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 shows the effects of N,N-dimethyl-p-phenylenediamine (DMPD) in regard to Aβ₄₀ aggregation under various conditions including Aβ₄₀ aggregation in the absence of metals or Aβ₄₀ aggregation induced by metals, and also shows the effects of DMPD in regard to cytotoxicity triggered by metal-free Aβ₄₀ or metal-treated Aβ₄₀;

FIG. 2 shows the interaction between DMPD and Aβ₄₀ monomer;

FIG. 3 shows the binding of DMPD to Zn(II);

FIG. 4 shows the results obtained by CuK-edge X-ray absorption spectroscopy analysis with respect to the interaction between Cu(I)-/Cu(II)-Aβ fibril and DMPD;

FIG. 5 shows the results obtained by UV-vis spectrophotometry for monitoring transformation of DMPD according to the presence or absence of Cu(II) or Aβ₄₀;

FIG. 6 shows the results obtained by mass spectrometry and ion mobility mass spectrometry with respect to the resulting products of the interaction between DMPD and Aβ₄₀;

FIG. 7 shows the results obtained by histopathology evaluation in terms of amounts of Aβ₄₀/Aβ₄₂ in the brain and of load of amyloid deposition after administering DMPD to a mouse; and

FIG. 8 shows the results obtained by reviewing improvements of cognitive impairment after administering DMPD to a mouse.

### DETAILED DESCRIPTION OF THE INVENTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. Expressions such as “at least one of,” when preceding a list of elements, modify the entire list of elements and do not modify the individual elements of the list.

Hereinafter, the present inventive concept will be described in further detail. According to the present inventive concept, there is provided a pharmaceutical composition for treating or preventing a degenerative brain disease, the pharmaceutical composition including, as an active component, a compound represented by Formula 1 or 2:

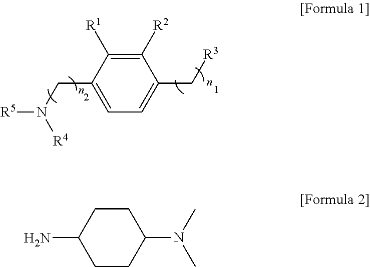

In Formula 1, R¹ to R³ may be identical to or different from each other, and may each be independently at least one of a hydrogen, a halogen atom, a di(C₁-C₄ alkyl)amino group, and a carboxyl group,

R⁴ and R⁵ may be identical to or different from each other, and may each be independently at least one of a hydrogen and a C₁-C₄ alkyl group, and

n₁ and n₂ may be identical to or different from each other, and may each be independently an integer of 0 or 1.

In Formula 1, R¹ to R² may each be at least one of a hydrogen, a halogen atom, and a di(C₁-C₄ alkyl)amino group, and R³ may be at least one of a hydrogen, a di(C₁-C₄ alkyl)amino group, and a carboxyl group.

In addition, in Formula 1, one substituent of R¹ to R³ may be selected from a dimethylamino group and a carboxyl group, and other remaining substituents of R¹ to R³ may be identical to or different from each other and are each independently separated from hydrogen and a halogen atom.

In addition, the compound of Formula 1 or 2 may be selected from N,N-dimethyl-p-phenylenediamine, N¹,N¹-dimethylbenzene-1,2-diamine, N¹,N¹-dimethylbenzene-1,3-diamine, 4-(aminomethyl)-N,N-dimethylaniline, 4-((dimethylamino)methyl)aniline, N¹,N¹-dimethyl-1,4-cyclohexanediamine, 4-(dimethylamino)benzoic acid, 4-aminobenzoic acid, 2-(4-(dimethylamino)phenyl)acetic acid, 2-(4-aminophenyl)acetic acid, 2-fluoro-N¹,N¹-dimethylbenzene-1,4-diamine, and 3-fluoro-N¹,N¹-dimethylbenzene-1,4-diamine.

The compound of Formula 1 or 2 may a gathering of amyloid-beta peptides in a manner of toxicity-free aggregation under conditions both in the presence or absence of metals such as copper and zinc, and also may react to multiple targets of Alzheimer's diseases at once to inhibit toxicity thereof, the multiple targets including amyloid-beta peptide, metal-amyloid-beta peptide, a metal, and an activated oxidizing species. In this regard, the pharmaceutical composition including the compound of Formula 1 or 2 may be utilized as a useful therapeutic agent or a health food in regard to a brain disease including Alzheimer's disease.

The degenerative brain disease may be one of Alzheimer's disease, Parkinson's disease, Lou Gehrig's disease, dementia, Huntington's disease, multiple sclerosis, amyotrophic lateral sclerosis, palsy, apoplexy, and mild cognitive impairment, and preferably, may be Alzheimer's disease.

The pharmaceutical composition according to the present inventive concept may further include suitable carriers, excipients, or diluents that are generally used in the preparation of pharmaceutical composition.

Carriers, excipients and diluents that can be contained in the composition according to the present invention include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil.

The pharmaceutical composition according to the present inventive concept may be formulated in the form of powders, granules, tablets, capsules, suspensions, emulsions, syrups, aerosols, and the like for oral applications, agents for external applications, suppositories, and sterile injection solutions, which are prepared according to methods known in the art.

When pharmaceutical composition according to the present inventive concept is formulated, diluents or excipients, such as fillers, extenders, binders, wetting agents, disintegrates or surfactants, which are commonly used in the art may be used. Solid formulations for oral administration include pills, powders, granules, capsules, and the like. Such solid formulations may be prepared by mixing the compound of the present inventive concept with at least one excipient, such as starch, calcium carbonate, sucrose, lactose, or gelatin.

In addition to simple excipients, lubricants such as magnesium stearate or talc may be also used. Liquid formulations for oral administration, such as suspensions, oral solutions, emulsions, syrups or the like, may include various excipients, such as wetting agents, sweeteners, aromatics, and preservatives, in addition to simple diluents, such as water and liquid paraffin.

Formulations for parenteral administration may include sterilized aqueous solutions, non-aqueous solvents, suspensions, emulsions, lyophilized agents, suppositories, or the like. Non-aqueous solvents and suspensions may be prepared using propylene glycol, polyethylene glycol, vegetable oils such as olive oil, or injectable esters such as ethyloleate. As a base for suppositories, Witepsol, Macrogol, Tween 61, cacao fat, laurin fat, or glycerogelatin may be used.

A dosage of the compound contained as the active component in the pharmaceutical composition according to the present inventive concept may differ according to a patient's age, gender, weight, or a type of disease. However, the compound contained as the active component in the pharmaceutical composition according to the present inventive concept may be administered in a range of about 0.001 to about 100 mg/kg, and preferably, about 0.01 to about 10 mg/kg in a single dose per day or in multiple doses per day.

In addition, a dosage of the compound contained as the active component in the pharmaceutical composition according to the present inventive concept may differ according to administration routes, severity of disease, and a patient's gender, weight, and age. Thus, the dosage is not intended to limit the scope of the present inventive concept in any way.

The pharmaceutical composition according to the present inventive concept may be administered to mammals including rats, mice, cattle, mammals, or humans, by various routes. Any administration method may be expected, and for example, the pharmaceutical composition according to the present inventive concept may be administered by oral, rectal or intravenous, muscular, subcutaneous, intrauterine, or intracerebroventricular injection, or endobronchotracheal inhalation.

In addition, according to the present inventive concept, there is provided a health food for preventing or improving a degenerative brain disease, the health food including, as an active component, a compound represented by Formula 1 or 2:

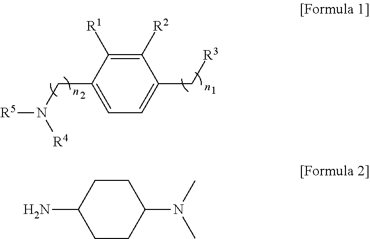

In Formula 1,

R¹ to R³ may be identical to or different from each other, and may each be independently at least one of a hydrogen, a halogen atom, a di(C₁-C₄ alkyl)amino group, and a carboxyl group,

R⁴ and R⁵ may be identical to or different from each other, and may each be independently at least one of a hydrogen and a C₁-C₄ alkyl group, and

n₁ and n₂ may be identical to or different from each other, and may each be independently an integer of 0 or 1.

The degenerative brain disease may be one of Alzheimer's disease, Parkinson's disease, Lou Gehrig's disease, dementia, Huntington's disease, multiple sclerosis, amyotrophic lateral sclerosis, palsy, apoplexy, and mild cognitive impairment, and preferably, may be Alzheimer's disease.

In addition, the health food according to the present inventive concept may be prepared in the form of powders, granules, tablets, capsules, syrups, or beverages. In addition to the compound contained as the active component in the health food according to the present inventive concept, other foods or food additives may be also used in an appropriate manner according to conventional methods known in the art. A mixing amount of the active component may be suitably determined by usage purposes, such as prevention, health or therapeutic treatment.

An amount of the active component contained in the health food according to the present inventive concept may be determined based on the effective dosage of the pharmaceutical composition according to the present inventive concept. However, in terms of health and hygiene, or in consideration of long-term administration for the purpose of health control, the amount of the active component may be less the range described above. Since there is nothing wrong with stability of the active component, it is certain that the active component may be used in an amount greater than the range described above.

Types of the health food according to the present inventive concept are not particularly limited, and examples thereof include meat, sausages, breads, chocolates, candies, snacks, cookies, pizza, Ramen or other noodles, gums, dairy products including ice cream, various soups, beverages, tea, drinks, alcohol drinks, and vitamin complex.

Hereinafter, the present inventive concept will be described in further detail with reference to the following examples. However, these examples are for illustrative purposes only and are not intended to limit the scope of the present invention.

### Reference Example

### Preparation of Reagents and Instruments

All reagents were commercially purchased, and unless specified otherwise, the purchased reagents were used as they were. For example, N,N-dimethyl-p-phenylenediamine (DMPD) was purchased from Sigma-Aldrich (St. Louis, Mo., USA), and Aβ₄₀ and Aβ₄₂ were purchased from AnaSpec (Fremont, Calif., USA) (Aβ₄₂=DAEFRHDSGYEVHHQKLVFFAEDVGSNKGAIIGLMVGGWIA).

An Agilent 8453 UV-visible (UV-vis) spectrometer (Santa Clara, Calif., USA) was used to measure optical spectra of the reagents, and the reagents were put in a N₂-filled glove box (Korea Kiyon, Bucheon-si, Gyeonggi-do, Republic of Korea) to allow an anaerobic reaction therein. Thermodynamic parameters of the reagents were measured using a VP isothermal titration calorimeter (MicroCal, Northampton, Mass., USA).

Transmission electron microscopic (TEM) images of the reagents were obtained by using a Philips CM-100 transmission electron microscope (Microscopy and Image Analysis Laboratory, University of Michigan, Ann Arbor, Mich., USA) or a JEOL JEM-2100 TEM (UNIST Central Research Facilities, Ulsan National Institute of Science and Technology, Ulsan, Republic of Korea).

Absorbance values of the reagents for cell viability assay were measured on a SpectraMax M5 microplate reader (Molecular Devices, Sunnyvale, Calif., USA).

A Bruker HCT basic system mass spectrometer equipped with electrospray ionization (ESI) ion source was used to obtain time-dependent mass spectra of DMPD that was incubated with Aβ.

All ion mobility-mass spectrometry (IM-MS) experiments were carried out on a Synapt G2 (Waters, Milford, Mass., USA).

NMR studies of DMPD in the presence or absence of Zn(II) were carried out on a 400 MHz Agilent NMR spectrometer, whereas NMR studies of DMPD and Aβ in the presence or absence of Zn(II) were carried out on a 900 MHz Bruker spectrometer equipped with a TCI triple-resonance inverse detection CryoProbe (Michigan State University, Lansing, Mich., USA).

### Example 1

### Analysis of DMPD Effects on Aβ Aggregation

1) Aβ Aggregation Experiments

Experiments with Aβ were conducted according to the methods that were already known in the art (Proc. Natl. Acad. Sci. USA 107, 21990-21995, 2010; Proc. Natl. Acad. Sci. USA 110, 3743-3748, 2013; J. Am. Chem. Soc. 136, 299-310, 2014; J. Am. Chem. Soc. 131, 16663-16665, 2009; Inorg. Chem. 51, 12959-12967, 2012).

Aβ₄₀ or Aβ₄₂ was dissolved in ammonium hydroxide (NH₄OH, 1% v/v aq), and then, aliquoted, lyophilized overnight, and stored at a temperature of −80° C. For experiments described herein, a stock solution of Aβ was prepared by dissolving lyophilized peptide in 1% NH₄OH (10 μL) and diluted with deionized distilled water (ddH₂O).

The concentration of Aβ peptides in the solution was determined by measuring the absorbance of the solution at 280 nm (E=1450 M⁻¹ cm⁻¹ for Aβ₄₀; and ε=1490 M⁻¹ cm⁻¹ for Aβ₄₂).

The peptide stock solution was diluted to a final concentration of 25 μM in Chelex-treated buffered solution containing [4-(2-hydroxyethyl)-1-piperazine ethane sulfonic acid; 20 μM; pH 6.6 for Cu(II) samples; pH 7.4 for metal-free and Zn(II) samples] (HEPES) and NaCl (150 μM).

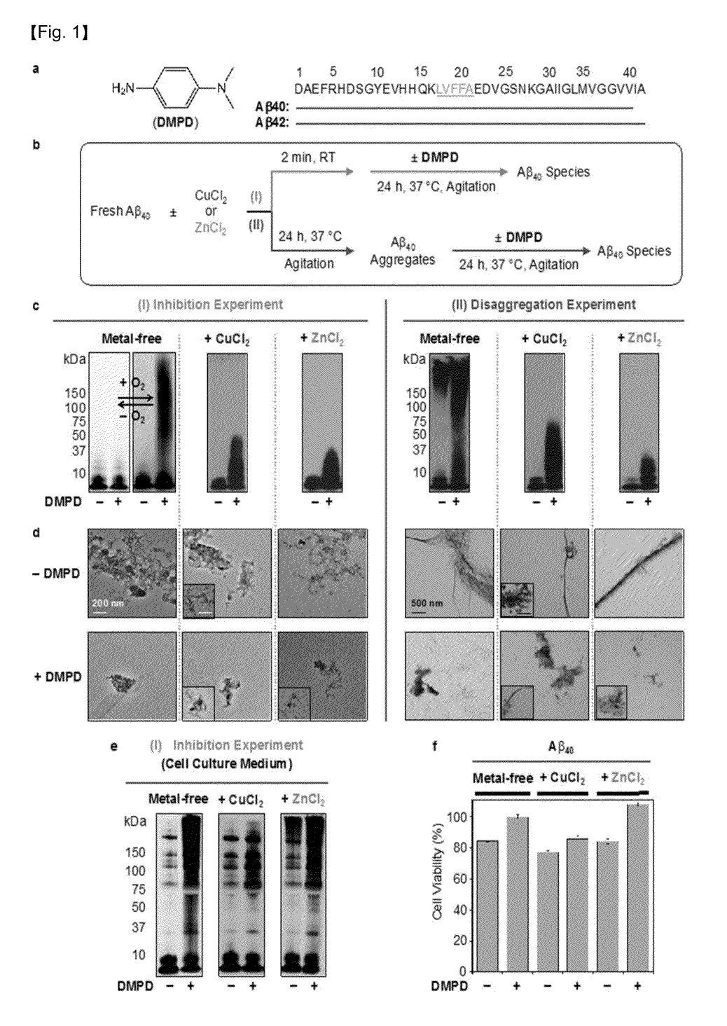

To review effects of DMPD on the inhibition of formation of Aβ aggregation, as shown in FIG. 1B (i), DMPD [50 μM; 1% v/v dimethyl sulfoxide (DMSO)] was added to the samples of Aβ (25 μM) in the presence or absence of a metal chloride salt (CuCl₂ or ZnCl₂, 25 μM), followed by incubation at a temperature of 37° C. with constant stirring for 24 hours.

To review effects of DMPD on the breakdown of Aβ aggregation, as shown in FIG. 1B (ii), Aβ in the presence or absence of a metal ion was stirred at a constant speed at a temperature of 37° C. for 24 hours, prior to the treatment of DMPD (50 μM).

2) Gel Electrophoresis and Western Blot

The samples from studies on the inhibition of formation of Aβ aggregation and the breakdown of Aβ aggregation were analyzed by gel electrophoresis with Western blotting using an anti-Aβ antibody (6E10).

Each sample (10 μL) was separated on a 10-20% Tris-tricine gel (Invitrogen, Grand island, NY, USA), and the protein samples obtained therefrom were transferred onto a nitrocellulose membrane which was blocked with bovine serum albumin (BSA, 3% w/v, Sigma-Aldrich, St. Louis, Mo., USA) in Tris-buffered saline containing 0.1% Tween-20 (TBS-T; 1.00 mM Tris base, pH 8.0, 1.50 mM NaCl) at room temperature for 2 hours.

Afterwards, the membranes were incubated with a primary antibody (6E10, Covance, Princeton, N.J., USA; 1:2000) in a solution of 2% w/v BSA (in TBS-T) overnight at 4° C. After washing with TBS-T (three times, each for 10 minutes), the horseradish peroxidase-conjugated goat antimouse secondary antibody (1:5000; Cayman Chemical Company, Ann Arbor, Mich., USA) in 2% w/v BSA (in TBS-T) was added for 1 hour at room temperature.

Afterwards, SuperSignal West Pico Chemiluminescent Substrate (Thermo Scientific, Rockford, Ill., USA) was used to visualize protein bands.

3) Transmission Electron Microscopy (TEM)

Glow-discharged grids (Formar/Carbon 300-mesh, Electron Microscopy Sciences, Hatfield, Pa., USA) were treated with Aβ samples from the inhibition and breakdown of Aβ aggregation experiments for 2 minutes.

Excess buffer was carefully removed by blotting with filter paper, and then, washed twice with ddH₂O. Each grid was incubated with uranyl acetate staining solution (1% w/v ddH₂O, 5 μL) for 1 minute. Excess stain was blotted off, and the grids were air dried at room temperature for at least 20 minutes.

Enlarged images from each sample were taken on a Philips CM-100 (80 kV) or a JEOL JEM-2100 TEM (200 kV) at a magnification of 25,000×.

4) Measurement of Cell Viability

The human neuroblastoma, i.e., SK-N-BE(2)-M17 (M17) cell line, was purchased from the American Type Culture Collection (ATCC, Manassas, Va., USA).

The cell line was maintained in media containing Minimum Essential Media (MEM; GIBCO, Life Technologies, Grand Island, N.Y., USA) and Ham's F12K Kaighn's Modification Media (F12K; GIBCO) at a ratio of 1:1, 10% (v/v) fetal bovine serum (FBS; Atlanta Biologicals, Flowery Branch, Ga., USA), 100 U/mL penicillin (GIBCO), and 100 mg/mL streptomycin (GIBCO). The cells were grown and maintained at a temperature of 37° C. in a humidified atmosphere with 5% CO₂.

M17 cells were loaded in a 96-well plate (15,000 cells in 100 μL) according to the methods known in the art. (Proc. Natl. Acad. Sci. USA 107, 21990-21995, 2010; J. Am. Chem. Soc. 131, 16663-16665, 2009).

These cells were treated with various concentrations of DMPD (0-10 μM, 1% v/v DMSO) in the presence or absence of CuCl₂ or ZnCl₂ (metal and ligand at a ratio of 1:1), and in the presence or absence of Aβ₄₀ (Aβ, a metal, and a ligand at a ratio of 10:10:20 μM).

After completing the incubation at a temperature of 37° C. for 24 hours, 25 μL of [3-(4,5-dimethylthiazol-2-yl)-2,5-diphenyltetrazolium bromide; 5 mg/mL in phosphate buffered saline (PBS), pH7.4, GIBCO] (MTT) was added to each well, and then, the plates were incubated at a temperature of 37° C. for 4 hours.

Formazan produced by the cells was dissolved in a solution containing N,N-dimethylformamide (DMF, 50% v/v aq, pH 4.5) and sodium dodecyl sulfate (SDS, 20% w/v) overnight at room temperature. Subsequently, absorbance at 600 nm was measured on a microplate reader.

5) Experiment Results

In the experiments of the inhibition of Aβ aggregation, different molecular weight (MW) distributions were observed for DMPD-treated Aβ₄₀/Aβ₄₂ in the presence or absence of metals compared to untreated analogues (see the left image of FIG. 1C). TEM images of FIG. 1 also revealed either small amorphous Aβ aggregates or shorter and more dispersed fibrils (see the left image of FIG. 1D). In the experiments of the breakdown of Aβ aggregation, DMPD indicated significant effects on the transformation of performed metal-free Aβ₄₀/Aβ₄₂ and metal-Aβ₄₀/Aβ₄₂ aggregates (see the right image of FIG. 1C), and moreover, DMPD also showed less reactivity toward Aβ₄₂ aggregates regardless of the presence or absence of metals. In TEM images of the DMPD-treated samples also revealed amorphous Aβ aggregates, shorter fibrils, or mixtures of two Aβ arrangements (see the right image of FIG. 1D).

Upon treatment of DMPD to Aβ₄₀ in a cell culture medium, a distinguishable variation in the MW distribution of Aβ species was still observed (see FIG. 1E). That is, in consideration of the formation of metal-free Aβ aggregates or metal-induced Aβ aggregates, it was confirmed that the treatment of DMPD inhibited the formation of Aβ aggregates, and modified the formed Aβ aggregates to relatively smaller and less structured Aβ aggregates.

In addition, viability was increased by about 10-20% when DMPD was introduced to Aβ₄₀- or metal-Aβ₄₀-treated M17 cells, relative to the untreated cells (see FIG. 1F).

### Example 2

### Analysis of Interactions Between Aβ and DMPD

The interactions between DMPD and metal-free Aβ were analyzed according to isothermal titration calorimetry (ITC), 2D NMR spectroscopy, and MD simulation.

1) Isothermal Titration Calorimetry (ITC) Analysis

ITC analysis was conducted with a solution of DMPD (200 μM, 10% v/v DMSO) and Aβ₄₀ (20 μM) dissolved in 20 mM HEPES (pH 7.4, 150 mM NaCl) was prepared as a ligand solution, and then, the ligand solution was degassed for 10 min prior to titration.

The ligand solution (10 μL) was titrated into the Aβ₄₀ solution (1.4 mL) at 25° C. over 1 second with 25 injections with a constant interval of 200 securing a 250 μL syringe rotating at 310 rpm. In a control experiment, the identical titrant solution was injected into the same buffer used with Aβ₄₀ to measure the heat of dilution.

A reasonable heat of binding value was calculated by subtracting the heat of dilution value from the overall heat change. Titration data were analyzed with the MicroCal Origin (v. 7.0). The values of Ka (association constant) and AH (binding heat exchanger) each ligand upon binding to Aβ₄₀ was determined from a proper fitting model. The binding curves were best fit to a sequential binding model with three binding sites. The values of −TΔS and ΔG were calculated from the Gibb's free energy relationship [(ΔG=ΔH−TΔS; ΔG=−RT ln(Ka)].

Thermodynamic parameters regarding the Aβ₄₀-DMPD interactions are shown in Table 1, and it was confirmed that the binding of Aβ₄₀-DMPD was significantly contributed to hydrophobic bindings in a thermodynamically advantageous manner.

TABLE 1

| Thermodynamic Parameters | Values |
|---|---|
| Kₐ₁ (M⁻¹) | 2.85 ± 0.88 × 10⁵ |
| Kₐ₂ (M⁻¹) | 4.34 ± 2.88 × 10⁴ |
| Kₐ₃ (M⁻¹) | 3.73 ± 2.22 × 10⁴ |
| ΔH₁ (kJ/mol) | −2.63 ± 0.35 |
| ΔH₂ (kJ/mol) | 4.79 ± 3.35 |
| ΔH₃ (kJ/mol) | −35.0 ± 6.37 |
| ΔG₁ (kJ/mol) | −31.1 ± 0.77 |
| ΔG₂ (kJ/mol) | −26.5 ± 0.16 |
| ΔG₃ (kJ/mol) | 26.1 ± 0.15 |
| TΔS₁ (kJ/mol) | 28.5 ± 0.84 |
| TΔS₂ (kJ/mol) | 31.3 ± 0.37 |
| TΔS₃ (kJ/mol) | −8.90 ± 0.65 |

2) 2D Band-Selective Optimized Flip-Angle Short Transient (SOFAST)-Heteronuclear Multiple Quantum Correlation (HMQC) NMR Spectroscopy

NMR titration experiments were performed according to the methods known in the art (Proc. Natl. Acad. Sci. USA 110, 3743-3748, 2013; J. Am. Chem. Soc. 136, 299-310, 2014; Chem. Commun. 50, 5301-5303, 2014).

NMR samples were prepared with ¹⁵N-labeled Aβ₄₀ (rPeptide, Bogart, Ga., USA) which was lyophilized in 1% NH₄OH by resuspending the peptide in 100 μL of 1 mM NaOH (pH 10).

To yield a final peptide concentration of 80 μM, the peptide was diluted with 200 mM phosphate buffer (pH 7.4), 1 M NaCl, D₂O, and water. Each spectrum was obtained using 256 complex t₁ points and a 1 second-recycle delay at a temperature of 4° C.

The 2D data were processed using TopSpin 2.1 (Bruker, Billerica, Mass., USA). Resonance assignments were carried out by computer-aided resonance assignment (CARA) using published assignments for Aβ (Biochem. Biophys. Res. Commun. 411, 312-316, 2011; Angew. Chem. Int. Ed. 50, 5110-5115, 2011).

Compiled chemical shift perturbation (CSP) was calculated using the following equation:

$${Δδ}_{NH} = \sqrt{( Δ δ {H}^{Z} + {( \frac{Δ δ N}{5} )}^{Z} )}$$

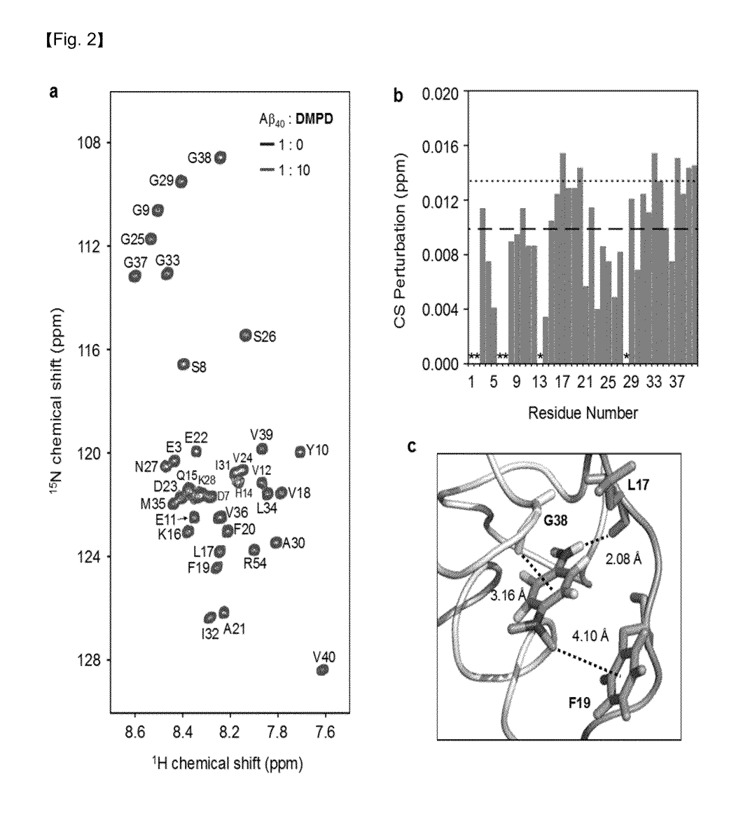

When DMPD was titrated into ¹⁵N-labeled Aβ₄₀, as shown in FIGS. 2A and 2B, relatively significant chemical shift perturbations (CSPs) were shown for six amino acid residues (particularly for L17, F20, G33, G37, V39, and V40). These residues correspond to the self-recognition (residues 17-21) and C-terminal hydrophobic regions, and are reported to be crucial for Aβ aggregation and cross b-sheet formation via hydrophobic interactions. The CSP presented for V40 may be due to intrinsic C-terminal disorder rather than interaction with DMOD. The distribution of observed CSPs suggests that DMPD could interact with the amino acid residues in Aβ₄₀ near the self-recognition and hydrophobic regions, and the suggestion is also supported by the thermodynamic data regarding the interactions of Aβ₄₀-DMPD.

3) Molecular Dynamics (MD) Simulations

To explore the interactions between Aβ₄₀ and DMPD, a multi-step computational strategy was used.

In the first step, 100 ns MD simulations in an aqueous solution were conducted to obtain the equilibrated structure of the Aβ₄₀ monomer. These simulations were performed using the GROMACS program (version 4.0.5) and GROMOS 96 53A6 force field. The starting structure of the Aβ₄₀ monomer was extracted from the NMR structures determined in aqueous SDS micelles at pH 5 (model 2, PDB 1BA4). The root-mean-square deviations (rmsd) indicated that the system reached the equilibration during the time frame of the simulations.

In the next step, to include the flexibility of the Aβ₄₀ monomer into the docking procedure, 100 snapshots were taken at 1 nanosecond (ns) interval throughout the simulation. These snapshots were used for the rigid docking of the DMPD molecule using the AutoDock Vina 1.1.2 software. In this procedure, the receptor was kept fixed, but the ligand was allowed to change its conformation. The DMPD molecule was built using the GaussView program (B3LYP/LanI3DZ), and then, optimized at the level of theory using the Gaussian03 program. In the docking procedure, the size of the grid was chosen to occupy the whole receptor-ligand complex. Each docking trial produced 20 poses with an exhaustiveness value of 20. The docking procedure provided 2000 poses. Based on binding energies and the composition of interacting sites, 20 distinct poses were selected for short-term (5 ns) MD simulations in an aqueous solution.

From these 20 different simulations, 5 structures were derived and further 20 ns simulations were performed using the same program and force field. These simulations provided a binding site that includes L17, F19, and G38 residues of the Aβ₄₀ monomer. The tools available in the GROMACS program package and the YASAFA software (v. 13.2.2) were utilized for analyzing trajectories and simulated structures.

For all simulations, the starting structures were placed in a truncated cubic box with dimensions of 7.0×7.0×7.0 nm. This dismissed unwanted effects that may arise from the applied periodic boundary conditions (PBC). The box was filled with single point charge (SPC) water molecules. Few water molecules were replaced by sodium and chloride ions to neutralize the system. The starting structures were subsequently energy-minimized with a steepest descent method for 3,000 steps. The results of the minimizations produced the starting structure for the MD simulations. The MD simulations were then carried out with a constant number of particles (N), pressure (P), and temperature (T)(i.e., NPT ensemble).

The SETTLE algorithm was used to constrain the bond length and angle of the water molecules, while the LINCS algorithm was used to constrain the bond length of the peptide. The Particle-Mesh Ewald (PME) method was implemented to treat the long-range electrostatic interactions. A constant pressure of 1 bar was applied with a coupling constant of 1.0 ps. The peptide, water molecules, and ions were coupled separately to a bath at 300 K with a coupling constant of 0.1 ps. The equation of motion was integrated at each 2 fs time steps using leap-frog algorithm.

As shown in FIG. 2C, the simulation results showed multiple interactions. That is, (i) a potential binding picket was formed through hydrogen bonding (2.08 Å) of the amine group of DMPD with an oxygen atom of the backbone carbonyl between L17 and V18, (ii) the aromatic ring of DMPD associated with G39 via a N-H-π interaction (3.16 Å), and (iii) the methyl group of the dimethyl amino group of DMPD stabilized the Aβ-DMPD interaction by the C-H-π (with the aromatic ring of F19) interaction (4.10 Å).

Thus, ITC, 2D NMR, and docking/MD simulation studies demonstrated the direct interaction of DMPD with metal-free Aβ species.

### Example 3

### Analysis of Interaction of DMPD with Metal-Aβ Monomers and Fibrils

1) Cu K-Edge X-Ray Absorption Spectroscopy (XAS)

Aβ₄₂ was monomerized according to the methods previously described (Biochemistry 44, 5478-5487, 2005; J. Am. Chem. Soc. 126, 13534-13538, 2004), and fibrils of Aβ₄₂ were grown according to protocols known in the art (Biochemistry, 47, 5006-5016, 2008). Following monomerizatino of fibrillization, all samples were treated under an anaerobic atmosphere (N₂) in a COY anaerobic chamber (COY Laboratory, Grass Lake, Mich., USA).

Aβ₄₂ was dissolved in a mixture of 10 mM of N-ethylmorpholine buffer (pH 7.4) and glycerol (used as a deicing agent) that were mixed at a ratio of 4:1, and then, an equivalent amount of CuCl₂ was added thereto. Aβ₄₂ monomers were maintained at 5° C., and all procedures performed rapidly to avoid aggregation. Following the addition of CuCl₂, 2 equivalent of ascorbate was treated with the resulting samples to reduce Cu(II)-loaded Aβ₄₂ peptides to Cu(I). Afterwards, DMPD (2 equivalents, dissolved in DMSO) was then introduces to each solution. Final Aβ₄₂ concentrations were 250 μM. DMPD was incubated with the copper-loaded fibrils for 24 hours. To avoid aggregation which was confirmed by gel permeation chromatography (GPC) studies, the copper-loaded Aβ₄₂ monomers were allowed to react with DMPD for 15 minutes.

Following the reaction with DMPD, the solutions were injected into Lucite sample holders with Kapton tape windows, and then, quickly frozen in liquid nitrogen. All data were recorded on beamline X-3b at the National Synchrotron Light Source (Brookhaven National Laboratories, Upton, N.Y., USA).

Samples were maintained at a temperature up to 18 K through data collection by means of a He Displex cryostat. Energy monochromatization was accomplished with a Si(111) double crystal monochromator and a low angle Ni mirror was used for harmonic rejection.

Data were collected as fluorescence spectra using a Canberra 31 element Ge solid-state detector with a 3 micron Ni filter placed between the sample and detector, and calibrated against a simultaneously collected spectrum of Cu-foil (first inflection point 8980.3 eV).

Count rates were between 15 and 30 kHz, and deadtime corrections yielded no improvement to the quality of the spectra.

Data were collected in 5 eV steps from 200-20 eV below the edge (averaged over 1 second), 0.5 eV steps from 20 eV below the edge to 30 eV above the edge (averaged over 3 seconds), 2 eV steps from 30-300 eV above the edge (averaged over 5 seconds), and 5 eV steps from 300 eV above the edge to 13 k (averaged over 5 seconds). Each data set represents the average of 16 individual spectra. Known glitches were removed from the averaged spectra. The X-ray beam was repositioned every 4 scans, and no appreciable photodamage/photoreduction was noted. Data were analyzed as previously reported using the software packages EXAFS123 and FEFF 7.02, wherein errors were reported as E² values.

2) Spectrophotometric Analysis

All samples were prepared in Chelex-treated buffered solution containing HEPES[20 μM; pH 6.6 for Cu(II) samples or pH 7.4 for metal-free and Zn(II) samples] and NaCl (150 μM). For Aβ-free samples, DMPD (50 μM) was treated with CuCl₂ or ZnCl₂ (25 μM) for 2 minutes. For Aβ-containing samples, Aβ₄₀ (25 μM) was treated with CuCl₂ or ZnCl₂ (25 μM) for 2 minutes, followed by the addition of DMPD. The absorption spectra of the resulting solution were obtained every 2 hours for 24 hours at room temperature without agitation. Metal free samples with and without Aβ₄₀ were also monitored by UV-vis in an anaerobic environment. All solvents required for the preparation of the anaerobic samples were degassed by freeze-pump-thaw cycling three times, and then, stored in a N₂-filled glove box. Anaerobic samples were prepared in a N₂-filled glove box. UV-vis spectra were recorded at 0, 4, and 24 hours of reaction time points at room temperature without agitation.

3) Mass Spectrometric Analysis

Samples containing DMPD (50 μM) and Aβ₄₀ (25 μM) for the experiment were prepared in 100 μL of 1 mM NH₄OAc (pH 7.4). The resulting solutions were allowed to react for 0, 2, 4, 8, and 24 hours at a temperature of 37° C. The samples were directly injected into the mass spectrometer at a flow rate of 240 mL/h. ESI interface was operated in positive-ion mode; spray voltage was set at 4.5 kV with capillary temperature at 300° C. and capillary exit voltage at 101 V. Mass spectra were taken in the range of m/z 50 to 500.

All ion mobility-mass spectrometry (IM-MS) experiments were carried out on a Synapt G2 (Waters, Milford, Mass., USA). Samples were ionized using a nano-electrospray source operated in positive ion mode. MS instrumentation was operated at a backing pressure of 2.7 mbar and a sample cone voltage of 40 V. Data were analyzed using MassLynx 4.1 and DriftScope 2.0 (Waters, Milford, Mass., USA). Collision cross-section (CCS) measurements were calibrated externally using a database of known protein, and protein complex CCS values in helium. Lyophilized Aβ₄₀ peptides (AnaSpec, Fremont, Calif., USA) were prepared to a stock concentration of 25 μM in 1 mM ammonium acetate (pH 7.0). Aliquots of Aβ₄₀ were then allowed to react with or without 50 μM DMPD (1% v/v DMSO) for 24 hours at a temperature of 25° C. without constant agitation. After the reaction, all samples were lyophilized overnight prior to re-suspension of the samples in hexafluoro-2-propanol (HFIP, Sigma-Aldrich, St Louis, Mo., USA)([Aβ₄₀]=50 μM) and sonicated under pulse settings for 5 minutes. The samples were diluted 50%, to a final Aβ₄₀ concentration of 25 μM, using 1 mM ammonium acetate (0.5 mM final concentration) immediately prior to mass analysis.

4) Experiments Results

i) Analysis of Binding of DMPD with Metal

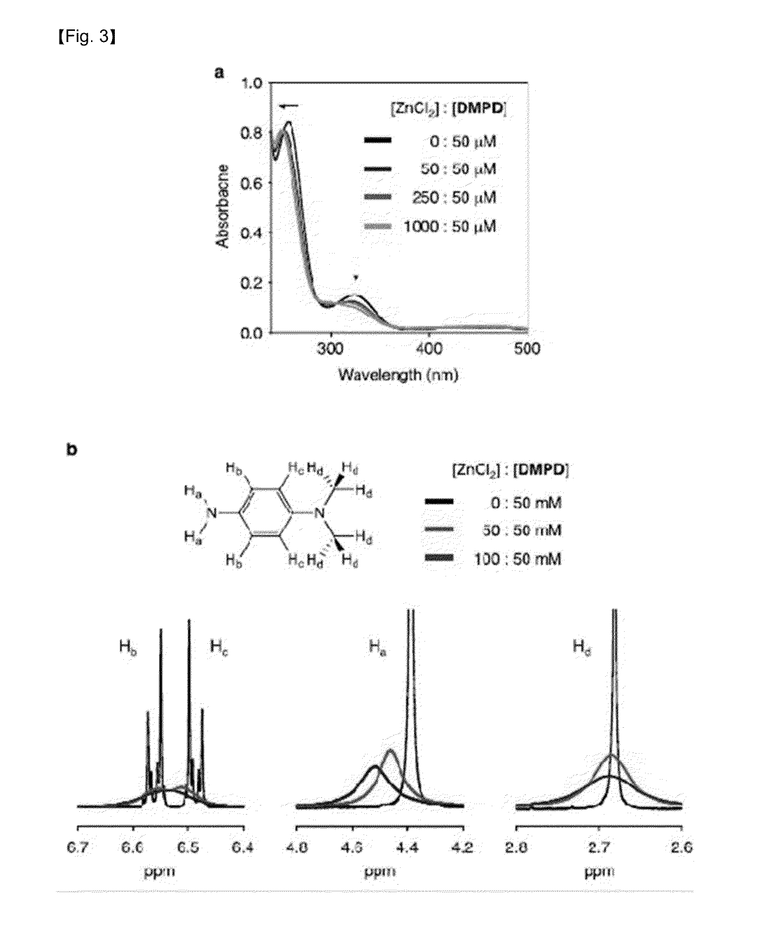

In regard to the binding between DMPD and Zn(II) through UV-vis spectra and ¹H NMR spectra, as shown in FIGS. 3A and 3B, it was confirmed that Zn(II) interacted with the N atom of the amino group of DMPD.

ii) Analysis of Binding of DMPD with Metal-Aβ Monomer and Fibrils

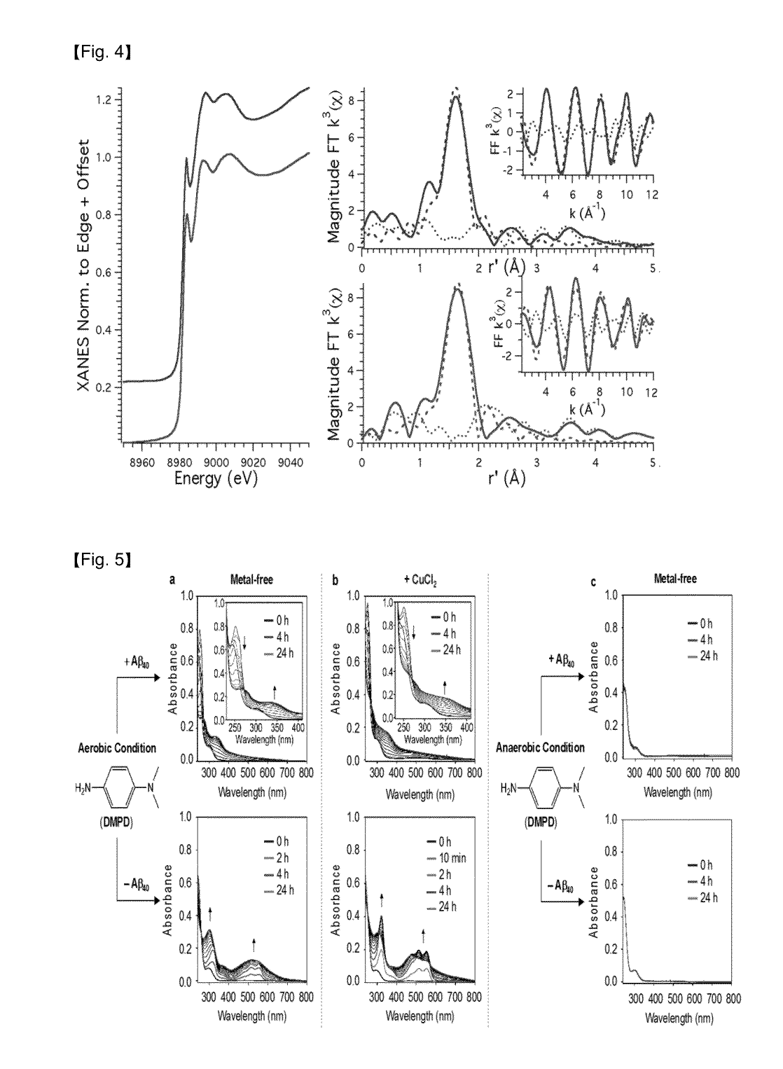

Following the treatment on DMPD, it was confirmed that the XAs data for Cu(I)-loaded Aβ₄₂ fibrils were consist with a linear 2-coordinate Cu(I)(N/O)₂ environment, as shown in FIG. 4 (red). The X-ray absorption near edge structure (XANES) region of the XAS spectrum exhibited a prominent pre-edge feature at 8985.2(2) eV corresponding to the Cu (1s→4p_{z}) transition. Such a feature is characteristic of linear Cu(I). As shown in FIG. 4 (blue), the Cu(II)-loaded Aβ₄₂ fibrils treated with DMPD indicated the complete reduction of Cu(II) to a linear 2-coordinate Cu(I)(N/O)₂ center. The observations from the XAS studies suggested interactions between copper-Aβ complexes and DMPD indicated the possible redox interaction on the basis of copper surrounded by DMPD and Aβ.

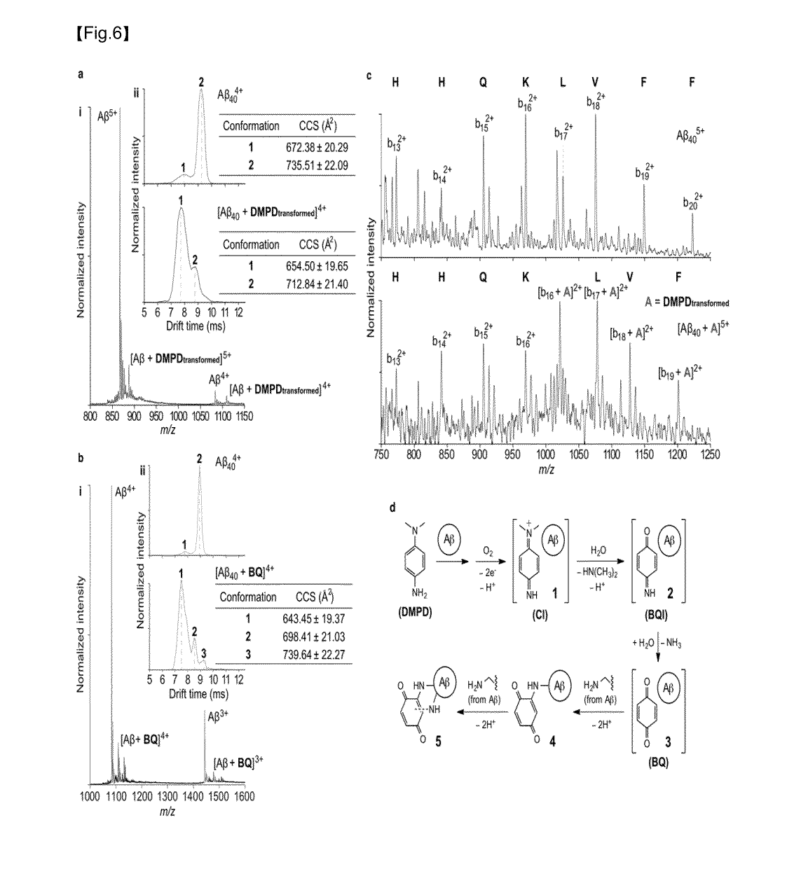

iii) Analysis of Mechanism of DMPD's Control Against Aβ Aggregation To examine the mechanism of DMPD's control against Aβ aggregation, the chemical transformation of DMPD with Aβ was analyzed under various conditions. As shown in FIGS. 5A to 5C, time-dependent optical changes of DMPD were monitored in the absence and presence of Aβ₄₀ with and without CuCl₂ in buffered solutions. Consequently, DMPD treated with Aβ₄₀ exhibited spectral shifts, different from the Aβ₄₀-free conditions (see FIGS. 5A and 5B). The optical bands at ca. 513 and 550 nm, indicative of the formation of a cationic radical of DMPD through an oxidative degradation route, were not observed even after a 24 hour reaction of DMPD with Aβ. Upon addition of DMPD into a solution containing Aβ₄₀, a shift in the optical band of DMPD immediately occurred from 295 to 305 nm. After a 4 hour reaction, strong bands at ca. 250 nm indicated isosbestic points at ca. 280 and 330 nm (see FIG. 5A, top). 4 Upon reaction over 4 h, a new optical band at ca. 340 or 350 nm with an isosbestic point at ca. 270 nm began to grow in (see FIGS. 5A and 5B, bottom). These optical bands at ca. 250 and 340 or 350 nm are expected to be indicative of generating a possible adduct of benzoquinoneimine (BQI) or benzoquinone (BQ) with proteins (see FIG. 6). Accordingly, DMPD could be transformed through a different pathway in the presence of Aβ, thereby producing a modified DMPD conjugate with Aβ.

In addition, to validate the involvement of oxygen in the conversion of DMPD, the UV-vis spectra of DMPD were measured in an anaerobic environment with or without Aβ. Consequently, as shown in FIG. 5C, spectral alterations of DMPD were not revealed in an anaerobic condition even after 24 hour reaction. Furthermore, modulation of Aβ aggregation by DMPD was not observed under the anaerobic condition (see FIG. 1C), distinguishable from that under the aerobic setting. That is, dioxygen is essential for the transformation of DMPD and Aβ peptides into less toxic aggregates.

In addition, as a result of MS analysis of the reaction between DMPD and Aβ for 0, 2, 4, 8, 24 hours, signals for DMPD at m/z 137 and signals for fragments produced by the loss of the amine groups at m/z 122 were decreased according to the reaction time. That is, the time-dependent decrease of the MS signals indicated that the interaction between DMPD and Aβ was produced by formation of conversion products.

Furthermore, to confirm the formation of Aβ₄₀-ligand complexes, the MS analysis was performed on the DMPD-treated Aβ₄₀ samples. Consequently, new peaks appeared upon the addition of 103.93±0.04 Da to Aβ (see FIG. 6A(i)), proposed to be referring to a covalently bound conversion product of DMPD (e.g., BQ).

To support our proposed mode of Aβ-DMPD interaction via the transformation of DMPD, the interactions of Aβ₄₀ with the structurally homologous BQ were examined under identical, experimental conditions. Consequently, it was confirmed that BQ binds to Aβ₄₀ (see FIG. 6b) with a mass shift that is consistent with DMPD incubations (104.1±0.1 Da). Tandem MS (MS/MS) in conjunction with collision induced dissociation (CID) for the 5⁺ ligand bound charge state was carried out to determine the nature of the Aβ₄₀-DMPD_{transformed} (see FIG. 6c). MS/MS data supports that both DMPD_{transformed} and BQ covalently link to Aβ₄₀ via K16 or K28. This ligated mass difference is too small to support a single covalent bond formation between Aβ and DMPD_{transformed}/BQ (106.1 Da expected), whereas it does agree well with the generation of a second covalent bond between Aβ and DMPD_{transformed}/BQ (104.1 Da expected). Thus, this data supports that DMPD_{transformed}/BQ is capable of crosslinking Aβ, which is consistent with the data previously published with the structurally homologous BQ examined under identical, experimental conditions. Consequently, it was confirmed that BQ binds to Aβ₄₀ (see FIG. 6b) with a mass shift that is consistent with DMPD incubations (104.1±0.1 Da). Tandem MS (MS/MS) in conjunction with collision induced dissociation (CID) for the 5⁺ ligand bound charge state was carried out to determine the nature of the Aβ₄₀-DMPD_{transformed} (see FIG. 6c). MS/MS data supports that both DMPD_{transformed} and BQ covalently link to Aβ₄₀ via K16 or K28. This ligated mass difference is too small to support a single covalent bond formation between Aβ and DMPD_{transformed}/BQ (106.1 Da expected), whereas it does agree well with the generation of a second covalent bond between Aβ and DMPD_{transformed}/BQ (104.1 Da expected). Thus, this data supports that DMPD_{transformed}/BQ is capable of crosslinking Aβ, which is consistent with the data previously published for a-synuclein (FEBS Journal 272, 3661-3672 (2005)).

In addition, IM-MS studies of the 4⁺ charge state were carried out in order to assess the Aβ-bound state conformers adopted. When compared to the apo state, Aβ-DMPD_{transformed} complexes possessed a significantly decreased ion mobility (IM) arrival time, indicative of a more compact Aβ₄₀ structure (see FIG. 6A (ii)). Consistent with this data, Aβ₄₀-BQ binding also leads to a similar reduction in IM arrival time, again supporting the production of a more compact species than the form adopted by the apopeptide (see FIG. 6B (ii)).

Combining this data with observations from our MS/MS analysis, it was determined that the DMPD_{transformed}/BQ crosslinking traps Aβ in a relatively compact conformational state that is likely off-pathway with respect to amyloid fibril formation.

In addition, based on these optical and MS results, it was determined that the Aβ-DMPD_{transformed} complexes could occur via two possible mechanism pathways, as shown in FIG. 6D.

In the presence of Aβ under aerobic conditions, DMPD could first undergo a two-electron oxidative transformation to generate a cationic imine (CI)-Aβ complex (i). CI could be converted via hydrolysis to BQI (ii) that could further hydrolyze its imine to generate BQ (iii). BQ is then capable of forming a covalently bound Aβ-BQ adduct through interactions with a primary amine containing residue (Aβ+106.1 Da, iv), such as K16, that further crosslinks to an additional residue with a similar functional group (Aβ+104.1 Da, v). The covalent complexation of Aβ with BQ could direct the structural compaction based on IM-MS analysis (see FIGS. 6A and 6B), and could account for DMPD's redirection of peptide aggregation pathways into amorphous Aβ aggregates, as found in the gel/Western blot and TEM studies.

### Example 4

### In Vivo Efficacy of DMPD Against Amyloid Pathology and Cognitive Impairment

To validate the beneficial effects of DMPD on AD pathogenesis in vivo, DMPD was administered to male 5×FAD mice via the intraperitoneal route at 1 mg/kg/day for 30 days from 3 months of age. After 30 total daily treatments of DMPD, mice were subjected to biochemical analysis for cerebral amounts of Aβ₄₀/Aβ₄₂ and histopathological evaluations of the amyloid deposition load. 5×FAD mice were selected for this study since they develop early and severe phenotypes of AD and behavioral dysfunction. At the conclusion of the DMPD treatment period, there was no significant difference in gross appearance or body weight between the vehicle- and DMPD-treated 5×FAD mice.

Cerebral Aβ peptides were quantified by the enzyme-linked immunosorbent assay (ELISA).

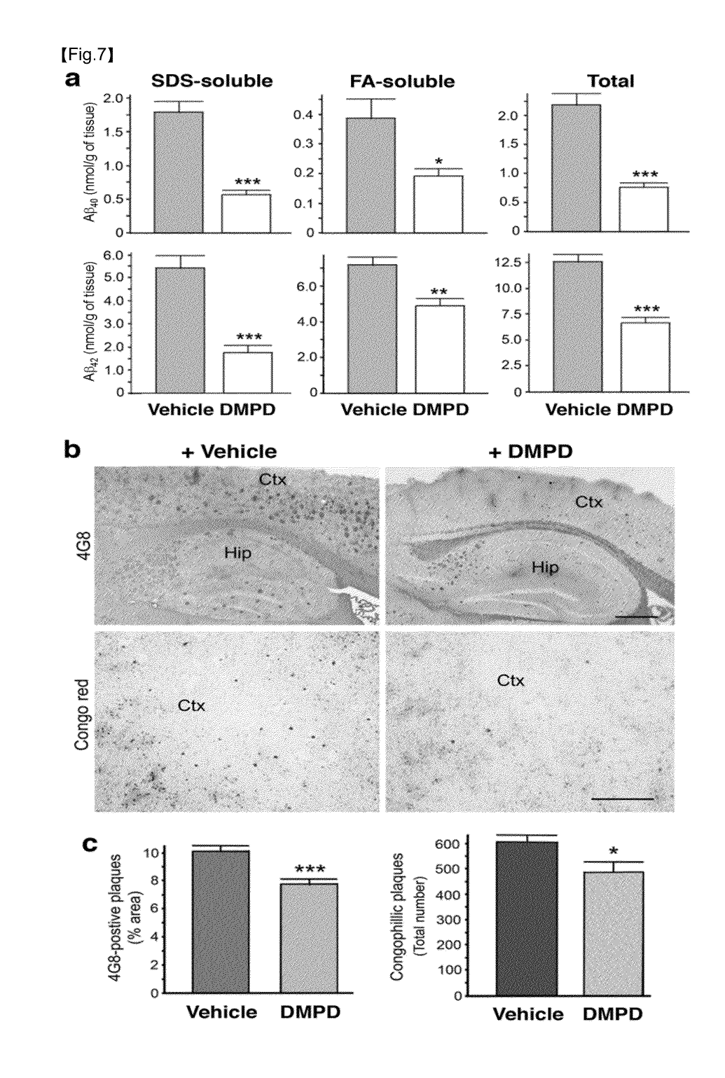

The ELISA results revealed that the total levels of Aβ₄₀/Aβ₄₂ containing PBS-, sodium dodecyl sulfate (SDS)-, and formic acid (FA)-soluble Aβ mice treated with DMPD were decreased by ca. 65% and 47%, respectively, compared to the vehicle-treated 5×FAD mice (see FIG. 7A).

The levels of SDS-soluble Aβ₄₀/Aβ₄₂ were more drastically reduced by DMPD (ca. 68% and 67%, respectively) than those of FA-soluble Aβ₄₀/Aβ₄₂ levels (ca. 50% and 32%, respectively). Furthermore, substantial reductions in amyloid deposits were detected in 5×FAD mice treated with DMPD, determined by analyzing the loads of amyloid precursor protein (APP))/Aβ-immunoreactive 4G8- and Congo red-stained amyloid plaques by ca. 23% and 20%, respectively (FIGS. 7B and 7C).

Overall, these results indicate that DMPD is able to delay or reverse the amyloid pathogenesis in the brain of the AD mouse model.

In addition, to evaluate the capacity of DMPD to improve cognitive impairment in AD model mice, 4-month-old 5×FAD mice were treated with DMPD, and then, the Morris water maze test for spatial learning and memory was performed during the final five consecutive days of the DMPD treatment.

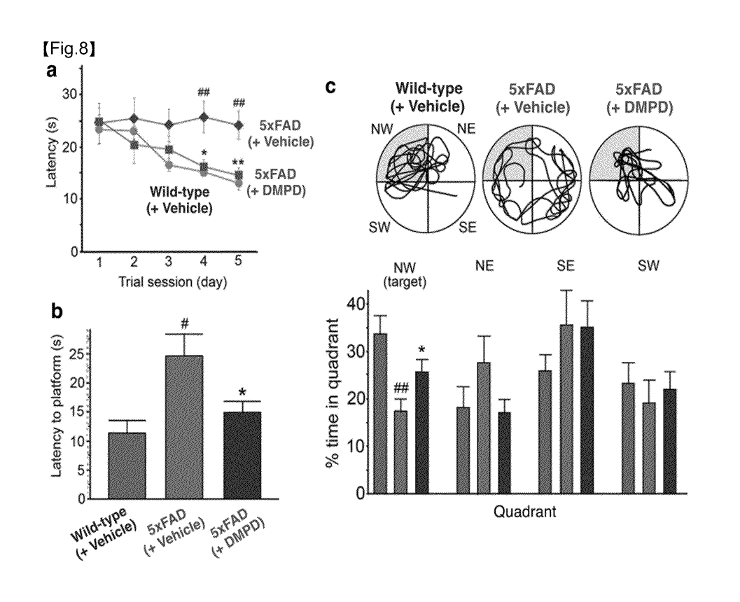

The 5×FAD mice exhibited impaired spatial learning, showing enhanced difficulty in locating the hidden escape platform in a pool of water compared to their littermate, wild-type mice. In contrast, the repetitive administration of DMPD prominently improved the learning and memory capability in the 5×FAD mice, relative to those of the vehicle-treated wild-type mice (see FIG. 8A).

Three hours after the final Morris water maze test, to confirm the performance of long-term memory retention, the probe trials, where the escape platform was removed, was carried out, and the mice located its previous position in the water for 60 seconds.

The DMPD-treated mice took distinguishably less time to reach the platform area and spent significantly more time in the target quadrant (North West, NW), where the platform had been hidden, than vehicle-treated 5×FAD mice (see FIGS. 8B and 8C).

Accordingly, it was confirmed that DMPD is capable of rescuing cognitive impairment in 5×FAD mice.

Hereinafter, preparation examples of DMPD-containing compositions will be described. However, these examples are for illustrative purposes only and are not intended to limit the scope of the present invention.

### Prescription Example 1

### Prescription Example of Pharmaceutical Compositions

### Prescription Example 1-1

### Preparation of Powders

20 mg of DMPD, 100 mg of lactose, and 10 mg of talc were mixed, and the mixture was filled in an airtight pack, thereby preparing powders.

### Prescription Example 1-2

### Preparation of Tablets

20 mg of DMPD, 100 mg of corn starch, 100 mg of lactose, and 2 mg of magnesium stearate were mixed, and the mixture was subjected to conventional preparation methods for compressed tablets known in the art, thereby preparing tablets.

### Prescription Example 1-3

### Preparation of Capsules

10 mg of DMPD, 100 mg of corn starch, 100 mg of lactose, and 2 mg of magnesium stearate were mixed, and the mixture was filled in gelatin capsules according to conventional preparation methods known in the art, thereby preparing capsules.

### Prescription Example 1-4

### Preparation of Injections

10 mg of DMPD, an appropriate amount of sterile distilled water, and an appropriate amount of pH controller were mixed, and the mixture was subjected to conventional preparation methods for injections known in the art, thereby preparing 2 mm per ampoule injections.

### Prescription Example 1-5

### Preparation of Ointment

10 mg of DMPD, 250 mg of PEG-4000, 650 mg of PEG-400, 10 mg of white Vaseline, 1.44 mg of methyl ρ-Hydroxybenzoate, 0.18 mg of propyl ρ-hydroxybenzoate, and remaining amounts of purified water were mixed, and the mixture was subjected to conventional preparation methods for ointment known in the art, thereby preparing ointment.

### Prescription Example 2

### Health Supplement Food

### Prescription Example 2-1

### Preparation of Health Food

1 mg of DMPD, an appropriate amount of vitamin mixtures (i.e., 70 μg of vitamin A acetate, 1.0 mg of vitamin E, 0.13 mg of vitamin B1, 0.15 mg of vitamin B2, 0.5 mg of vitamin B6, 0.2 μg of vitamin B12, 10 mg of vitamin C, 10 μg of biotin, 1.7 mg of nicotinamide, 50 μg of folate, and 0.5 mg of calcium pantothenate), and an appropriate amount of inorganic mixtures (i.e., 1.75 mg of ferrous sulfate, 0.82 mg of zinc oxide, 25.3 mg of magnesium carbonate, 15 mg of monopotassium phosphate, 55 mg of calcium hydrogen phosphate, 90 mg of potassium citrate, 100 mg of calcium carbonate, and 24.8 mg of magnesium chloride) were mixed, and the mixture was subjected to conventional preparation methods for granules known in the art, thereby preparing health food.

### Prescription Example 2-2

### Preparation of Health Drink

1 mg of DMPD, 1,000 mg of citric acid, 100 g of oligosaccharides, 2 g of pulm extract, 1 g of taurine, and purified water were mixed to a total amount of 900 mL according to conventional preparation methods for health drink known in the art. Then, the mixed solution was heat-stirred at a temperature of 85° C. for 1 hour, and then, filtered. The filtrate was then obtained in a sterilized 2 L-container that was sequentially sealed and sterilized to be stored under refrigeration.

As described above, according to one or more of the above exemplary embodiments, regarding a pharmaceutical composition for treating or preventing a degenerative brain disease, the pharmaceutical composition including, as an active component, a multi-targeting compound for further improving treatment efficiency of a degenerative brain disease such as Alzheimer's disease, the compound according to present inventive concept induces a gathering of amyloid-β peptides in a manner of toxicity-free aggregation under conditions both in the presence and absence of metals such as copper and zinc, and also reacts to multiple targets of Alzheimer's diseases at once to inhibit toxicity thereof, the multiple targets including amyloid-β peptide, metal-amyloid-β peptide, a metal, and an activated oxidizing species. In this regard, the compound to present inventive concept may be utilized as a useful therapeutic agent or a health food in regard to a brain disease including Alzheimer's disease.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

1. A method of treating or preventing a degenerative brain disease in a subject in need thereof, comprising:
providing a pharmaceutical composition comprising a compound represented by Formula 1 or 2 as an active components, 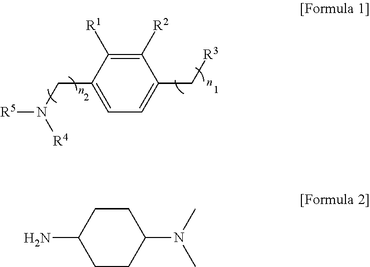
and
administering the pharmaceutical composition to the subject, wherein the degenerative brain disease is treated or prevented,
wherein, in Formula 1,
R¹ and R³ are identical to or different from each other, and are each independently at least one of a hydrogen, a halogen atom, a di(C₁-C₄ alkyl)amino group, and a carboxyl group,
R⁴ and R⁵ are identical to or different from each other, and are each independently at least one of a hydrogen and a C₁-C₄ alkyl group, and
n₁ and n₂ are identical to or different from each other, and are each independently an integer of 0 or 1.

2. The method of claim 1, wherein, in Formula 1, R¹ and R² are each independently at least one of a hydrogen, a halogen atom, and a di(C₁-C₄ alkyl)amino group, and R³ is at least one of a hydrogen, a di(C₁-C₄ alkyl)amino group, and a carboxyl group.

3. The method of claim 1, wherein, in Formula 1, one substituent of R¹ to R³ is selected from a dimethylamino group and a carboxyl group, and other remaining substituents of R¹ to R³ are identical to or different from each other and are each independently separated from a hydrogen and a halogen atom.

4. The method of claim 1, wherein the compound of Formula 1 or 2 is selected from N,N-dimethyl-p-phenylenediamine, N¹,N¹-dimethylbenzene-1,2-diamine, N¹,N¹-dimethylbenzene-1,3-diamine, 4-(aminomethyl)-N,N-dimethylaniline, 4-((dimethylamino)methyl)aniline, N¹,N¹-dimethyl-1,4-cyclohexanediamine, 4-(dimethylamino)benzoic acid, 4-aminobenzoic acid, 2-(4-(dimethylamino)phenyl)acetic acid, 2-(4-aminophenyl)acetic acid, 2-fluoro-N¹,N¹-dimethylbenzene-1,4-diamine, and 3-fluoro-N¹,N¹-dimethylbenzene-1,4-diamine.

5. The method of claim 1, wherein the compound of Formula 1 or 2 induces a gathering process of amyloid-peptides in a manner of toxicity-free aggregation under conditions in the presence or absence of a metal.

6. The method of claim 1, wherein the compound of Formula 1 or 2 reacts to multiple targets of Alzheimer's diseases at once to inhibit toxicity thereof, the multiple targets including amyloid-peptide, metal-amyloid-peptide, a metal, and an activated oxidizing species.

7. The method of claim 1, wherein the generative brain disease is one of Alzheimer's disease, Parkinson's disease, Lou Gehrig's disease, dementia, Huntington's disease, multiple sclerosis, amyotrophic lateral sclerosis, palsy, apoplexy, and mild cognitive impairment.

8. A method of preventing or improving a degenerative brain disease in a subject in need thereof, comprising:
providing a health food comprising a compound represented by Formula 1 or 2 as an active component, 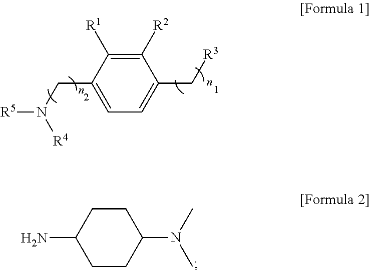
and
administering the health food to the subject, wherein the degenerative brain disease is prevented or improved,
wherein, in Formula 1,
R¹ to R³ are identical to or different from each other, and are each independently at least one of a hydrogen, a halogen atom, a di(C₁-C₄ alkyl)amino group, and a carboxyl group,
R⁴ and R⁵ are identical to or different from each other, and are each independently at least one of a hydrogen and a C₁-C₄ alkyl group, and;
n₁ and n₂ are identical to or different from each other, and are each independently an integer of 0 or 1.

9. The method of claim 8, wherein the generative brain disease is one of Alzheimer's disease, Parkinson's disease, Lou Gehrig's disease, dementia, Huntington's disease, multiple sclerosis, amyotrophic lateral sclerosis, palsy, apoplexy, and mild cognitive impairment.
